(19) United States
(12) Patent Application Publication
(10) Pub. No.: US 2004/0022855 A1
(43) Pub. Date: Feb. 5, 2004

(54) Cored tablets comprising amoxicillin and clavulanate

(75) Inventors: Dong-Jin Yoon, Yongin-Shi (KR); Min-Suk Lee, Seoul (KR); Young-Ghil Shin, Yongin-Shi (KR)

Correspondence Address: Peter F. Corless, Dike, Bronstein, Roberts & Cushman, IP Group of, EDWARDS & ANGELL, LLP, 101 Federal Street, Boston, MA, 02110-1800, US

(73) Assignee: Daewoong Pharm Co., Ltd., Republic of Korea

(21) Appl. No.: 10/630,557

(22) Filed: Jul. 29, 2003

(30) Foreign Application Priority Data
Aug. 1, 2002 (KR) 2002-45585

(51) Int. Cl. A61K 31/397; A61K 31/43; A61K 31/407; A61K 9/24

(52) U.S. Cl. 424/471; 514/210.09; 514/192

(57) ABSTRACT

The present invention provides a cored tablet comprising a core layer containing clavulanate, and an outer layer containing amoxicillin and surrounding the core layer, and a method for preparing the same.

### TECHNICAL FIELD

The present invention relates to a cored tablet comprising amoxicillin and clavulanate. Specifically, the cored tablet comprises a core layer containing clavulanate and an outer layer surrounding the core layer and containing amoxicillin.

### BACKGROUND ART

Amoxicillin is a semisynthetic broad-spectrum β-lactam antibiotic, and clavulanate is an inhibitor of β-lactamase, which antagonizes β-lactamase mediated resistance. Thus, clavulanate is used together with amoxicillin to enhance pharmacological effects of amoxicillin. A combined formulation of amoxicillin and clavulanate is marketed under the trade name of Augmentin™.

Currently, the combined formulation of amoxicillin and clavulanate is marketed in various dosage forms such as film-coated tablets, chewable tablets, suspensions, and the like, but the most frequent form thereof is the film-coated tablet or tablet form.

Clavulanate is very sensitive to moisture, and so rapidly decomposes in the presence of moisture. According to the Guidelines for Antibiotic Drug Products provided by the Korea Food and Drug Administration (KFDA) Rule, the moisture of amoxicillin itself is 11.5 to 14.5%, that of potassium clavulanate itself is less than 1.5%, and that of a preparation containing amoxicillin and potassium clavulanate in the ratio of 2:1 is 7.5 to 9.5%. Therefore, the moisture of potassium clavulanate is 1.5% alone, but that of the admixture with amoxicillin is much higher as 7.5 to 9.5%. Due to such high moisture, potassium clavulanate in admixture with amoxicillin rapidly decomposes during storage to have a problem of storage-stability.

The U.S. Pat. No. 6,051,255 discloses a method for manufacturing tablets comprising the steps of: compressing a mixture of amoxicillin and clavulanate; and film-coating the compressed mixture. Korean Laid-open No. 99-87104 discloses a method for producing a formulation containing amoxicillin and clavulanate, comprising the steps of: forming pastes of amoxicillin with a liquid solution; drying the pastes to obtain auxiliary-free aggregates having a mean particle size of 100 to 1000 μm, mixing the aggregates with clavulanate, and compressing the mixture by the direct powder method. However, in the above disclosed methods, clavulanate contacts with amoxicillin, resulting in increase of its moisture. Further, clavulanate is unavoidably exposed to the exterior, and so the content of the active ingredient is decreased during storage, resulting in decrease of its stability.

### DISCLOSURE OF THE INVENTION

The present inventors have extensively studied to develop a stable oral formulation of amoxicillin and clavulanate that can be prepared by a simple method as well as can prevent clavulanate from decomposing. As a result, the present inventors found that in case of preparing a combined formulation of amoxicillin and clavulanate in the form of cored tablet, comprising a core layer containing clavulanate, and an outer layer containing amoxicillin and surrounding the core layer, clavulanate can be prevented from contacting with amoxicillin, and further, the amoxicillin-containing outer layer perfectly blocks moisture from the outside, thereby to stabilize clavulanate in the core layer, and thus, the above formulation has an improved pharmacological effect. Therefore, the present invention was completed.

The present invention provides a cored tablet comprising a core layer and an outer layer surrounding the core layer, wherein the core layer contains clavulanate, and the outer layer contains amoxicillin. The cored tablet separates clavulanate from amoxicillin by containing clavulanate in the core layer and amoxicillin in the outer layer, respectively, thereby to prevent moisture increase caused by mixing clavulanate with amoxicillin as well as moisture input from the outside, and thereby to minimize the moisture content of the core layer containing clavulanate.

In a preferable embodiment of the present invention, amoxicillin is in the form of trihydrate, and clavulanate is in the form of salt with an alkali metal, for example, potassium clavulanate.

In another preferable embodiment of the present invention, the core layer is film-coated to more perfectly separate clavulanate from amoxicillin and thereby to minimize the moisture content of the core layer containing clavulanate. In a further preferable embodiment, the outer layer is film-coated to minimize moisture input from the outside.

The structure of the cored tablet according to the present invention is exemplified in FIGS. 1 and 2.

As shown in FIG. 1, in the cored tablet of the present invention, the core layer containing clavulanate is positioned in the center of the tablet, and the outer layer containing amoxicillin is in the outer side of the tablet in surrounding the core layer.

FIG. 2 is a sectional view of a preferable embodiment of the present invention. In FIG. 2, the core layer and the outer layer are film-coated to perfectly separate clavulanate from amoxicillin and to block moisture input from the outside.

In the cored tablet of the present invention, the weight-by-weight ratio of amoxicillin by clavulanate is preferably in the range of 1:1 to 7:1, and more preferably, 2:1.

The present invention also provides a method for preparing a cored tablet comprising a core layer containing clavulanate, and an outer layer containing amoxicillin and surrounding the core layer, comprising the steps of:

(i) compressing a mixture of clavulanate and one or more pharmaceutically acceptable carriers to obtain the core layer; and

(ii) introducing the obtained core layer into a mixture of amoxicillin and one or more pharmaceutically acceptable carriers, and compressing the whole mixture to obtain the outer layer.

A preferable embodiment of the present method further comprises the step of film-coating the core layer obtained in step (i). Another preferable embodiment of the present method further comprises the step of film-coating the outer layer obtained in step (ii).

A preferable example for the method of the present invention is as follows.

Clavulanate is mixed with one or more pharmaceutically acceptable carriers in a conventional mixer, and the mixture is compressed by the direct power method or dry granulation method to obtain a core layer. The obtained core layer is film-coated with a conventional film-coating solution. The film-coated core layer is introduced into a manufacturer of double core tablets, and then, amoxicillin is mixed with one or more pharmaceutically acceptable carriers, and the mixture is compressed into tablets again to obtain an outer layer having the central core layer, thereby to complete the present cored tablet. Subsequently, the outer layer is film-coated.

The cored tablet according to the present invention may comprise one or more pharmaceutically acceptable carriers, for example, excipients, binders, disintegrators, lubricants, colorants, and the like.

The excipient which may be used in the present invention is a conventional one, and preferably, is selected from the group consisting of lactose, microcrystalline cellulose, low-substituted hydroxypropyl cellulose, corn starch, potato starch, wheat starch, sucrose, glucose, fructose, D-mannitol, precipitated calcium carbonate, dextrin, methylcellulose, and mixtures thereof. The excipient is preferably present in an amount of 10 to 90% by weight based on the total weight of the tablet.

The binder which may be used in the present invention is a conventional one, and preferably, is selected from the group consisting of polyvinyl pyrrolidone, hydroxypropyl cellulose, microcrystalline cellulose, hydroxypropyl methyl cellulose, dextrin, gelatin, methyl cellulose, hydroxyl cellulose, hydroxymethyl cellulose, polyvinyl alcohol, and mixtures thereof. The binder is preferably present in an amount of 2 to 40% by weight of the total weight of the tablet.

The disintegrator which may be used in the present invention is a conventional one, and preferably, is selected from the group consisting of sodium starch glycollate, crospovidone, sodium croscamellose, low-substituted hydroxypropyl cellulose, hydroxypropyl methyl cellulose, polyvinyl pyrrolidone, starch, calcium carboxymethyl cellulose, ethyl cellulose, and mixtures thereof. The disintegrator is preferably present in an amount of 0.1 to 30% by weight of the total weight of the tablet.

The lubricant which may be used in the present invention is a conventional one, and preferably, is selected from the group consisting of magnesium stearate, talc, stearic acid, light anhydrous silicic acid, and mixtures thereof. The lubricant is preferably present in an amount of 0.1 to 20% by weight of the total weight of the tablet.

If necessary, a colorant may be used in the present invention, whose examples are one or more selected from the group consisting of food blue No. 1 aluminum lake, food red No. 40 aluminum lake, and food yellow No. 203 aluminum lake. The colorant may be added at an appropriate amount.

A coating agent used for film-coating the core layer and the outer layer of the present cored tablet is a conventional one, and preferably, is selected from the group consisting of methyl cellulose, hydroxypropyl cellulose, hydroxypropyl methyl cellulose, hydroxypropyl methyl cellulose phthalate, ethyl cellulose, carboxymethyl ethyl cellulose, polyethylene glycol, methacrylate copolymer, and mixtures thereof. The coating agent is preferably present in an amount of 0.5 to 10% by weight of the total weight of the tablet.

A coating solution may contain a plasticizer such as propylene glycol, myvacet, glycerol, sorbitol, glycerol triacetate, diethyl phthalate, and triethyl citrate; and a colorant such as titanium dioxide pigment, or iron oxide pigment, in addition to the above coating agent. Such additional agents facilitate the coating process and improve the shape of the coated film.

The dosages of amoxicillin and clavulanate to the human body may be appropriately selected depending on absorption rate, inactivation rate, and excretion rate in vivo, and the patient's age, sexuality, and condition, and also severity of diseases. For example, a unit dosage form for adults contains 250 mg of amoxicillin and 125 mg of clavulanate when administering three times a day.

### BRIEF DESCRIPTION OF THE DRAWINGS

FIG. 1 is a perspective view of the cored tablet according to the present invention.

FIG. 2 is a sectional view of one embodiment of the cored tablet according to the present invention.

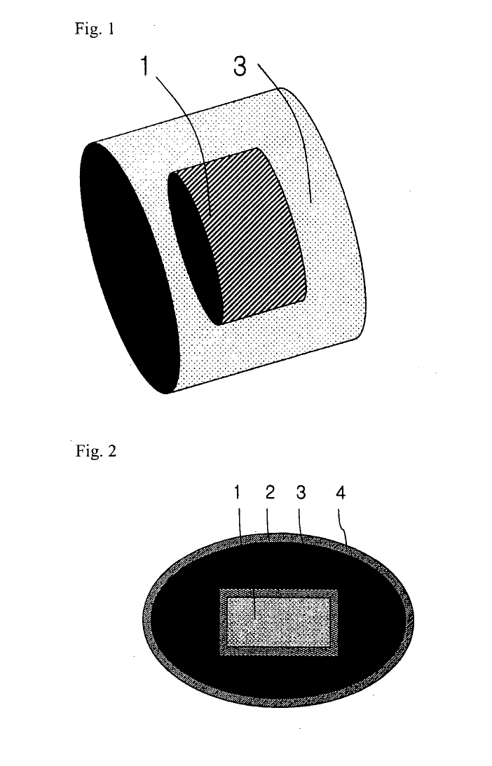

| | |
|---|---|
| 1: Core layer; | 2: Film-coating layer |
| 3: Outer layer; | 4: Film-coating layer |

### BEST MODE FOR CARRYING OUT THE INVENTION

The present invention will be more specifically illustrated by the following examples. However, the following examples should not be construed as limiting the scope of the present invention in any way.

### EXAMPLE 1

A. Preparation of a Core Layer Containing Clavulanate

(1) Preparation of a core layer

| | | |
|---|---|---|
| Core layer | 154.0 | mg per tablet |
| Potassium clavulanate (as the active ingredient) | 125.0 | mg |
| Microcrystalline cellulose | 20.0 | mg |
| Hydroxypropyl cellulose | 2.0 | mg |
| Calcium carboxymethyl cellulose | 5.0 | mg |
| Magnesium stearate | 2.0 | mg |

The above ingredients except magnesium stearate were mixed in a V-type mixer for 20 minutes, and the mixture was granulated using a roller compressor (Sejong Machine) at the roller speed of 5 to 10 rpm and the screw speed of 5 to 10 rpm. The granules were sieved using #12 to #16 mesh screen. To the obtained granules was added magnesium stearate for 5 minutes, and the mixture was compressed using Killian tablet machine at 10 to 30 rpm.

(2) Film-coating of a core layer

| | | |
|---|---|---|
| Hydroxypropyl methyl cellulose | 5.0 | mg |
| Titanium dioxide | 1.0 | mg |
| Talc | 0.5 | mg |
| Polyethylene glycol | 0.5 | mg |

The above ingredients were added to a suitable amount of ethanol to prepare a film-coating solution. The core layer of clavulanate obtained in the above A-(1) was film-coated with the coating solution by using HCT-48 coating machine (Freund) under the condition of the temperature of 25-30° C., the fan rotation speed of 4-5 rpm, the air pressure of 6 to 7 bar, and the relative humidity of 27% or less, to obtain the film-coated core layer of clavulanate.

B. Preparation of an Outer Layer Containing Amoxicillin

(1) Preparation of an outer layer

| | | |
|---|---|---|
| Outer layer | 308.0 | mg per tablet |
| Amoxicillin trihydrate (as the active ingredient) | 250.0 | mg |
| Microcrystalline cellulose | 40.0 | mg |
| Hydroxypropyl cellulose | 4.0 | mg |
| Calcium carboxymethyl cellulose | 10.0 | mg |
| Magnesium stearate | 4.0 | mg |

The above ingredients except magnesium stearate were mixed in a V-type mixer for 20 minutes, and the mixture was granulated using a roller compressor (Sejong Machine) at the roller speed of 5 to 10 rpm and the screw speed of 5 to 10 rpm. The granules were sieved using #12 to #16 mesh screen. To the obtained granules was added magnesium stearate for 5 minutes, and then, the mixture was further mixed with the clavulanate core layer obtained in the above A. The whole mixture was compressed at the rotary disk speed and the tabletting speed of 10 to 30 rpm to obtain the cored tablet containing the core layer.

(2) Film-coating of an outer layer

| | | |
|---|---|---|
| Hydroxypropyl methyl cellulose | 20.0 | mg |
| Titanium dioxide | 4.0 | mg |
| Talc | 8.0 | mg |
| Polyethylene glycol | 2.0 | mg |

The above ingredients were added to a suitable amount of ethanol to prepare a film-coating solution. The cored tablet obtained in the above B-(1) was film-coated with the coating solution by using HCT-48 coating machine (Freund) under the condition of the temperature of 25-30° C., the fan rotation speed of 4-5 rpm, the air pressure of 6 to 7 bar, and the relative humidity of 27% or less, to obtain the film-coated cored tablet.

### EXAMPLE 2

A. Preparation of a Core Layer Containing Clavulanate

(1) Preparation of a core layer

| | | |
|---|---|---|
| Core layer | 100.0 | mg per tablet |
| Potassium clavulanate (as the active ingredient) | 62.5 | mg |
| Vivapur ™ 12 | 30.5 | mg |
| Hydroxypropyl cellulose | 3.0 | mg |
| Light anhydrous silicic acid | 2.0 | mg |
| Magnesium stearate | 2.0 | mg |

The core layer was prepared according to substantially the same method as in the above Example 1-A-(1).

(2) Film-coating of a core layer

| | | |
|---|---|---|
| Hydroxypropyl methyl cellulose | 5.0 | mg |
| Titanium dioxide | 2.0 | mg |
| Ethyl cellulose | 1.0 | mg |
| Diethyl phthalate | 0.8 | mg |

The above ingredients were added to a suitable amount of water to prepare a film-coating solution. Then, the film-coated core layer of clavulanate was prepared according to substantially the same method as in the Example 1-A-(2).

B. Preparation of an Outer Layer Containing Amoxicillin

(1) Preparation of an outer layer

| | | |
|---|---|---|
| Outer layer | 200.0 | mg per tablet |
| Amoxicillin trihydrate (as the active ingredient) | 125.0 | mg |
| Vivapur ™ 12 | 63.0 | mg |
| Hydroxypropyl cellulose | 6.0 | mg |
| Light anhydrous silicic acid | 4.0 | mg |
| Magnesium stearate | 2.0 | mg |

The cored tablet was prepared according to substantially the same method as in the above Example 1-B-(1).

(2) Film-coating of a cored tablet

| | | |
|---|---|---|
| Hydroxypropyl methyl cellulose | 10.0 | mg |
| Titanium dioxide | 4.0 | mg |
| Ethyl cellulose | 2.0 | mg |
| Diethyl phthalate | 1.6 | mg |

The above ingredients were added to a suitable amount of water to prepare a film-coating solution. Then, the film-coated cored tablet was prepared according to substantially the same method as in the Example 1-B-(2).

### EXAMPLE 3

A. Preparation of a Core Layer Containing Clavulanate

(1) Preparation of a core layer

| | | |
|---|---|---|
| Core layer | 100.0 | mg per tablet |
| Potassium clavulanate (as the active ingredient) | 62.5 | mg |
| Ludipress ™ | 30.5 | mg |
| Hydroxypropyl cellulose | 3.0 | mg |
| Light anhydrous silicic acid | 2.0 | mg |
| Magnesium stearate | 2.0 | mg |

The core layer was prepared according to substantially the same method as in the above Example 1-A-(1).

(2) Film-coating of a core layer

| | | |
|---|---|---|
| Hydroxypropyl methyl cellulose | 7.0 | mg |
| Hydroxypropyl methyl cellulose phthalate | 2.0 | mg |
| Polyethylene glycol 6000 | 1.0 | mg |

The above ingredients were added to a mixed solution of ethanol and methylene chloride to prepare a film-coating solution. Then, the film-coated core layer of clavulanate was prepared according to substantially the same method as in the Example 1-A-(2).

B. Preparation of an Outer Layer Containing Amoxicillin

(1) Preparation of an outer layer

| | | |
|---|---|---|
| Outer layer | 300.0 | mg per tablet |
| Amoxicillin trihydrate (as the active ingredient) | 125.0 | mg |
| Ludipress ™ | 163.0 | mg |
| Hydroxypropyl cellulose | 6.0 | mg |
| Light anhydrous silicic acid | 4.0 | mg |
| Magnesium stearate | 2.0 | mg |

The cored tablet was prepared according to substantially the same method as in the Example 1-B-(1).

(2) Film-coating of a cored tablet

| | | |
|---|---|---|
| Hydroxypropyl methyl cellulose | 10.0 | mg |
| Hydroxypropyl methyl cellulose phthalate | 4.0 | mg |
| Ethyl cellulose | 4.0 | mg |
| Polyethylene glycol 6000 | 2.0 | mg |

The above ingredients were added to a mixed solution of ethanol and methylene chloride. Then, the film-coated cored tablet was prepared according to substantially the same method as in the Example 1-B-(2).

### COMPARATIVE EXAMPLE 1

A. Preparation of a naked tablet

| | | |
|---|---|---|
| Naked tablet | 439.0 | mg per tablet |
| Potassium clavulanate (as the active ingredient) | 125.0 | mg |
| Amoxicillin trihydrate (as the active ingredient) | 250.0 | mg |
| Microcrystalline cellulose | 30.0 | mg |
| Hydroxypropyl cellulose | 6.0 | mg |
| Calcium carboxymethyl cellulose | 20.0 | mg |
| Magnesium stearate | 8.0 | mg |

The naked tablet was prepared according to substantially the same method as in the above Example 1-A-(1).

B. Outer film-coating

| | | |
|---|---|---|
| Hydroxypropyl methyl cellulose | 20.0 | mg |
| Titanium dioxide | 4.0 | mg |
| Talc | 8.0 | mg |
| Polyethylene glycol | 2.0 | mg |

The above ingredients were added to a suitable amount of ethanol to obtain a film-coating solution, and then, the film-coated tablet was prepared according to substantially the same method as in the Example 1-A-(2).

### COMPARATIVE EXAMPLE 2

A. Preparation of a naked tablet

| | | |
|---|---|---|
| Naked tablet | 307.5 | mg per tablet |
| Potassium clavulanate (as the active ingredient) | 62.5 | mg |
| Amoxicillin trihydrate (as the active ingredient) | 125.0 | mg |
| Vivapur ™ 12 | 100.0 | mg |
| Hydroxypropyl cellulose | 12.0 | mg |
| Light anhydrous silicic acid | 5.0 | mg |
| Magnesium stearate | 3.0 | mg |

The naked tablet was prepared according to substantially the same method as in the above Example 1-A-(1).

B. Outer film-coating

| | | |
|---|---|---|
| Hydroxypropyl methyl cellulose | 10.0 | mg |
| Titanium dioxide | 4.0 | mg |
| Ethyl cellulose | 2.0 | mg |
| Diethyl phthalate | 1.6 | mg |

The above ingredients were added to a suitable amount of water to obtain a film-coating solution, and then, the film-coated tablet was prepared according to substantially the same method as in the Example 1-A-(2).

### COMPARATIVE EXAMPLE 3

A. Preparation of a naked tablet

| | | |
|---|---|---|
| Naked tablet | 307.5 | mg per tablet |
| Potassium clavulanate (as the active ingredient) | 62.5 | mg |
| Amoxicillin trihydrate (as the active ingredient) | 125.0 | mg |
| Ludipress ™ | 100.0 | mg |
| Hydroxypropyl cellulose | 12.0 | mg |
| Light anhydrous silicic acid | 5.0 | mg |
| Magnesium stearate | 3.0 | mg |

The naked tablet was prepared according to substantially the same method as in the above Example 1-A-(1).

B. Outer film-coating

| | | |
|---|---|---|
| Hydroxypropyl methyl cellulose | 10.0 | mg |
| Hydroxypropyl methyl cellulose phthalate | 4.0 | mg |
| Ethyl cellulose | 4.0 | mg |
| Polyethylene glycol 6000 | 2.0 | mg |

The film-coated tablet was prepared according to substantially the same method as in the Example 1-A-(2).

### EXPERIMENTAL EXAMPLE 1

### Stability Test of Clavulanate and Amoxicillin

According to the Stability Test Guidelines of the KFDA Rule No. 2000-7, an accelerated test to store samples under the condition of 40° C. and 75% relative humidity was performed for the respective twenty film-coated tablets prepared in the above Examples 1 to 3 and Comparative Examples 1 to 3 to measure the contents of amoxicillin and clavulanate. The content measurement was carried out according to the quantitation method of ‘amoxicillin-potassium clavulanate tablet’ listed in the Guidelines for Antibiotic Drug Products under the following column and conditions:

i) Column: Capcell-pak C18 UG120 (4.6 mm×25 cm, 5 μm, shiseido)

ii) Mobile Phase: Buffer solution* of pH 6.0

The buffer solution was prepared by dissolving ammonium formate of 6.3 g in 750 ml of water to adjust its pH to 6.0 by adding formic acid or ammonia, and thereto were added 30 ml of methanol and water to make the final volume of 1 l.

iii) Flow rate: 1 ml/min

iv) Detection wavelength: ultraviolet absorptive spectrophotometer (230 nm)

The average contents of clavulanate and amoxicillin are shown in the following Table 1.

TABLE 1
Contents of amoxicillin and clavulanate in the film-coated tablets (%)

| | | Examples | | | Comparative Examples | | |
|---|---|---|---|---|---|---|---|
| | | 1 | 2 | 3 | 1 | 2 | 3 |
| Clavulanate | 0 m | 100.3 | 100.5 | 99.8 | 100.1 | 99.7 | 100.5 |
| | 1 m | 99.8 | 99.5 | 98.9 | 93.7 | 92.5 | 90.4 |
| | 2 m | 99.7 | 98.7 | 97.8 | 85.9 | 86.3 | 84.5 |
| | 4 m | 97.8 | 96.5 | 96.1 | 78.5 | 79.8 | 76.8 |
| Amoxicillin | 0 m | 101.2 | 100.8 | 99.7 | 99.9 | 100.7 | 101.1 |
| | 1 m | 100.2 | 99.7 | 98.7 | 96.4 | 97.2 | 98.6 |
| | 2 m | 98.9 | 98.2 | 97.6 | 94.7 | 93.6 | 96.7 |
| | 4 m | 98.1 | 97.1 | 96.4 | 90.6 | 89.7 | 92.5 |

m: Month

As shown in the above Table 1, the film-coated tablets of Examples 1 to 3 contained clavulanate more than 96%, i.e. 97.8%, 96.5%, and 96.1%, after the storage of 4 months. By contrast, the film-coated tablets of Comparative Examples 1 to 3 contained clavulanate of much less contents, 78.5%, 79.8%, and 76.8%. In addition, the film-coated tablets of Examples 1 to 3 contained almost the same content of amoxicillin, but those of Comparative Examples 1 to 3 contained much less contents of amoxicillin. Therefore, it was confirmed that the film-coated tablets of the present invention have much higher stability than the prior film-coated tablets.

### INDUSTRIAL APPLICABILITY

The cored tablet comprising a core layer containing clavulanate, and an outer layer containing amoxicillin and surrounding the core layer according to the present invention can improve stability of the active ingredients, particularly, clavulanate.

What is claimed is:

1. A cored tablet comprising a core layer and an outer layer surrounding the core layer, wherein the core layer contains clavulanate, and the outer layer contains amoxicillin.

2. The cored tablet of claim 1, wherein the core layer is film-coated.

3. The cored tablet of claim 1, wherein the outer layer is film-coated.

4. The cored tablet of claim 1, wherein the weight-by-weight ratio of amoxicillin and clavulanate is in the range of 1:1 to 7:1.

5. The cored tablet of claim 4, wherein the weight-by-weight ratio of amoxicillin and clavulanate is 2:1.

6. A method for preparing the cored tablet of claim 1, comprising the steps of:
(i) compressing a mixture of clavulanate and one or more pharmaceutically acceptable carriers to obtain a core layer; and
(ii) introducing the obtained core layer into a mixture of amoxicillin and one or more pharmaceutically acceptable carriers, and compressing the mixture to obtain an outer layer.

7. The method of claim 6, further comprising the step of film-coating the core layer obtained in step (i) of claim 6.

8. The method of claim 6, further comprising the step of film-coating the outer layer obtained in step (ii) of claim 6.

9. The method of claim 6, wherein the weight-by-weight ratio of amoxicillin and clavulanate is in the range of 1:1 to 7:1.

10. The method of claim 9, wherein the weight-by-weight ratio of amoxicillin and clavulanate is 2:1.
